(19) United States
(12) Patent Application Publication
(10) Pub. No.: US 2006/0115527 A1
(43) Pub. Date: Jun. 1, 2006

(54) CONTROLLED RELEASE PREPARATION

(76) Inventors: Emadeldin M. Hassan, Greensboro, NC (US); Nachiappan Chidambaram, High Point, NC (US); Aqeel A. Fatmi, Greensboro, NC (US)

Correspondence Address: WOMBLE CARLYLE SANDRIDGE & RICE, PLLC, ATTN: PATENT DOCKETING 32ND FLOOR, P.O. BOX 7037, ATLANTA, GA, 30357-0037, US

(21) Appl. No.: 11/275,563

(22) Filed: Jan. 17, 2006

Related U.S. Application Data
(63) Continuation of application No. PCT/US04/22456, filed on Jul. 14, 2004.
(60) Provisional application No. 60/487,968, filed on Jul. 17, 2003.

(51) Int. Cl. A61K 31/522; A61K 9/52; A61K 31/195; A61K 31/415; A61K 31/192

(52) U.S. Cl. 424/457; 514/263.31; 514/406; 514/567; 514/570; 514/569

(57) ABSTRACT

Controlled release preparations and capsules are provided. Also provided are emulsions and suspensions, including compositions and methods of manufacturing controlled release capsules, where the fill contains a suspension and/or an emulsion.

### FIELD OF THE INVENTION

This invention relates generally to controlled release preparations and soft capsules. The invention relates further to emulsions and suspensions, including compositions and methods of manufacturing controlled release capsules where the fill contains a suspension and/or an emulsion.

### BACKGROUND OF THE INVENTION

Controlled release preparations have been a vital development in healthcare sciences. One advantage of such medicaments is improved patient compliance, especially where patients are under multiple or chronic treatments. Regarding the need to increase compliance rates, it is noted that the growing population of elder people further increases the demand for controlled release medication. Elderly patients often have particular difficulty with compliance for multiple daily dosages, especially in the context of a multiplicity of required medications.

While patient compliance is an immediate benefit of controlled release products, minimization of side effects of potent medicines is also a desirable advantage of controlled release preparations. For example, tachycardia, a well-known side effect of the cardiovascular drug nifidipine, can be significantly controlled when the drug is administered in a controlled release form. In fact, using controlled release preparations helps avoid sudden high drug concentrations of drugs in the systemic circulation and reduces subsequent adverse effects or toxicity.

Oral controlled release technologies are classified generally as of “matrix” or “film” nature. The matrix type is mainly used in tablets using polymeric or lipid materials that control both penetration of water and the release of the active ingredient to the surrounding environment. For example, U.S. Pat. No. 4,882,167 describes tablet compositions containing a hydrophobic carbohydrate polymer, e.g. ethyl cellulose and a wax material such as carnauba wax and made by direct compression. Despite of the apparent simplicity of the direct compression technique, it has limitations when applied to low dose, potent active ingredients. The low amounts of potent drugs are hardly well distributed in a directly compressed matrix due to the uncontrolled differences in particle size and density between the drug and matrix particles. Such differences usually lead to lack of homogeneous distribution of the drug in the matrix and lack of content uniformity. To overcome the limitations of direct compression matrix manufacture, a wet granulation technique is often applied. An example of the wet granulation procedure is described in U.S. Pat. No. 6,572,889 to Guo where granulation of active materials such as cabamazepine is performed in presence of water and polymeric substances. While wet granulation basically improves the distribution of an active material in a matrix, it is still considered a tedious and time-consuming technique.

The second major technology for oral controlled release preparations is applying coating or films to control the drug release from particles (e.g., pellets or microcapsules) or unit doses such as tablets. U.S. Pat. Nos. 5,871,776 and 4,572,833 provide details of preparing controlled release particles that can be filled into hard gelatin capsules or compressed into tablets. While pellets or microcapsules are fairly popular in controlled release products, they are considered an intermediate product that requires additional manufacturing steps to produce as a useful dosage form suitable for direct consumption by patients. On the other hand, coating unit doses such as tablets seems to be a more direct approach to manufacture oral controlled release pharmaceuticals. Tablet coating for controlled release purposes has been quite well known in the pharmaceutical industry for a long period of time and is well illustrated in standard pharmaceutical text books (see for example Remington's Pharmaceutical Industries, 18^{th} edition, Pages 1666 to 1675. Alfonso Gennaro, editor, Mack Publishing Co. Easto, Pa., 1990). As experienced persons in the art would expect, unit dose coating has many drawbacks that may lead to performance failures due to defects in the coat, such as pinholes and sticking.

Soft capsules have been tested as a controlled drug delivery system by Cohen, et al. (U.S. Pat. No. 4,795,642), where an aqueous fill of the polysaccharide gum sodium alginate forms a gel in presence of cationic elements such as heavy metal ions. However, the manufacture of soft capsules is presently the least utilized technique for producing oral controlled release preparations.

### SUMMARY OF THE INVENTION

The present invention provides numerous matrix systems based on lipids and lipophilic materials either alone or in presence of a hydrophilic phase. The described matrices have a hydrophobic surface in contact with the hydrophilic capsule shell to minimize any potential shell-fill interactions, as described elsewhere when soft capsules are filled with hydrophilic materials such as polyethylene glycol or similar vehicles.

This invention provides compositions of controlled release products and methods of preparation thereof. The present invention also provides compositions and methods of manufacture of controlled release medicaments in the soft gel dosage form. The invention also provides methods for manufacture of the fill of a controlled release soft gel in the form of a suspension, where part or all of the active ingredient or drug is suspended or dissolved in a matrix. Also provided are compositions and methods where the active ingredient or drug of a medicament is incorporated in a one-phase or two-phase matrix. A one-phase matrix can be comprised of homogeneous lipid materials, while the two-phase matrix can comprise an emulsion of aqueous hydrophilic material as the internal phase, and a hydrophobic external phase.

Accordingly, in one aspect the invention relates to a controlled release soft capsule having a shell and a matrix fill, wherein the matrix fill includes an active ingredient or drug incorporated as solid particles in lipid or lipophilic materials. In some embodiments, the lipid or lipophilic material can be a vegetable oil, hydrogenated vegetable oil, fatty acid, wax, fatty acid ester, or a combination thereof. The matrix fill can include a release regulator which can be a fatty acid salt, fatty acid ester, or fatty acid polyoxyethylene derivative. The release regulator can be a surfactant having an hydrophilic/lipophilic balance (HLB) value between about 3 and about 40.

In some embodiments, the active ingredient or drug can be a non-steroid anti-inflammatory drug or an anti-asthmatic. The active ingredient or drug can be diclofenac, naproxene, ibuprofen, ketoprofen, celecoxib, or theophylline. The ratio of the active ingredient or drug to the matrix fill can be from about 1:9 to about 1:1 by weight. The ratio can also be from about 1:8 to about 1:1 by weight.

In another aspect, the invention relates to a controlled release soft capsule having a shell and a matrix fill including an active ingredient or drug, wherein the physical state of the matrix can be a semi-fluid, or a structured solid state. In some embodiments, the matrix can be a fluid or semi-fluid at room temperature, or at a body temperature of a subject to which the capsule is intended to be administered. In some embodiments, the active ingredient or drug can be partially soluble in the matrix and at least a portion of the active ingredient or drug can be in solid form in the matrix.

In another aspect, the invention relates to a controlled release soft capsule including a shell and a matrix fill, wherein the matrix fill includes two phases in the form of an emulsion. In some embodiments, the emulsion can be a water-in-oil type emulsion. The emulsion can include a surfactant or combination of surfactants having HLB values ranging from about 2 to about 20. The HLB values can also range from about 5 to about 15.

In some embodiments, the active ingredient or drug can be an anti-asthmatic, narcotic analgesic, narcotic antagonist, or cardiovascular drug. The active ingredient or drug can be diltiazem, nifidipine, oxycodone, morphine, morphine analogues, or morphine antagonists.

In some embodiments, the ratio of the active ingredient or drug to the matrix fill can be from about 1:100 to about 1:2 by weight. The ratio can also be from about 1:50 to about 1:3 by weight.

In some embodiments, the emulsion can include an aqueous or hydrophilic internal phase and a lipid or lipophilic external phase. The internal phase can include polyethylene glycol of molecular weight ranging from about 200 to about 8000. In some embodiments, the internal phase can be an aqueous or hydro-alcoholic solution including cellulose derivatives, polyacrylates, polyvinyl polymers, or combinations thereof.

In some embodiments, the internal phase can include at least one polymer which can be methylcellulose, hydroxypropylmethyl cellulose, polymethylmethacrylate, or polyvinylpyrrolidone (PVP). The internal phase can also be structured.

In some embodiments, the external phase can include a vegetable oil, hydrogenated vegetable oil, fatty acid, wax, fatty acid ester, or a combination thereof.

In some embodiments, the active ingredient or drug can be dispersed in the internal phase as a solution or suspension form.

In some embodiments, the ratio of the internal phase to external phase can be from about 0.5:10 to about 1:1 by weight. The ratio can also be from about 1:9 to about 1:1 by weight.

In another aspect, the invention relates to a controlled release soft capsule having a shell and a matrix fill, wherein the matrix fill includes two phases in the form of an emulsion, with an active ingredient or drug distributed in both an external and internal phase. The active ingredient or drug can be in the form of solid particles. The active ingredient or drug can be present as solid particles incorporated in both the internal phase and the external phase.

In another aspect, the invention relates to a method of manufacturing a matrix fill for a controlled release soft capsule according to the invention. The method includes applying heat to the matrix components during mixing or prior to mixing at about the melting point of the matrix fill composition; and mixing the active ingredient or drug with the lipid or lipophilic matrix ingredients using mechanical or ultrasonic forces to form the matrix fill. The matrix fill can be flowable such that it can be encapsulated using a rotary die encapsulation machine. In some embodiments, the matrix components can be heated to a temperature in the range of from about 25° C. to about 70° C. The matrix components can also be heated to a temperature in the range of from about 30° C. to about 50° C.

In another aspect, the invention relates to a method of manufacturing a controlled release soft capsule, wherein the matrix fill includes two phases in the form of an emulsion. The method includes dispersing the active ingredient or drug in an internal phase to form a clear solution or suspension using propeller or homogenizer mixers; adding the internal phase materials to a molten external phase containing at least one surfactant in an amount from about 0.1% to about 5% by weight to form a resulting mixture; forming an emulsion from the resulting mixture by subjecting the mixture to mechanical forces generated by a propeller mixer, a homogenizer, or a microfluidizer; cooling the emulsion to from about 20° C. to about 35° C.; and encapsulating the emulsion using a rotary die encapsulation machine to form the controlled release capsule.

In yet another aspect, the invention relates to controlled release hard shell capsules containing the matrix fills of the invention. In particular embodiments, the matrix fills of the invention are encapsulated in two-piece hard shell capsules.

### DETAILED DESCRIPTION OF THE INVENTION

Generally, the controlled release soft capsules according to the invention comprise a shell and a matrix fill. The matrix fill can be a suspension-type matrix or an emulsion-type matrix.

In an embodiment of the invention having a suspension-type matrix fill, the active ingredient or drug is incorporated in the matrix fill as solid particles in lipid or lipophilic materials such as vegetable oils, hydrogenated vegetable oils, fatty acids, waxes, or fatty acid esters, or a combination thereof. The matrix composition may further contain a release regulator to modify the release profile to suit an optimum therapeutic requirement. The release regulator can be a surface-active agent that enhances water penetration into the lipid or lipophilic matrix to increase drug release. Examples of release regulators are fatty acid slats, fatty acid esters, or fatty acid polyoxyethylene derivatives. Surfactants having HLB values between about 3 and about 40 can be selected as release regulators. The hydrophilic/lipophilic balance (HLB) characteristic of surfactants can be determined in accordance with “Physical Pharmacy: Physical Chemical Principles in the Pharmaceutical Sciences,” Fourth Edition, pp. 371-373, A. Martin, Ed., Lippincott Williams & Wilkins, Philadelphia (1993).

In another embodiment of the invention having a suspension-type matrix fill, the matrix, at room or body temperature, can be in a fluid or structured solid state (solid, semi-solid, or gel). The drug can be partially soluble in the matrix while the rest of the drug is in a solid form. The presence of drug in two physical forms, solid particles and solution, can be useful by providing dual release patterns where one drug state is released faster than the other form.

In addition to suspension-type matrix fills, the invention also includes emulsion-type fills. Such fills are described herein as “emulsion-type” fills because they comprise an emulsion. The matrix fills for these embodiments can be characterized generally as emulsion-type fills, even though the active ingredient or drug can be present as a suspension in one or more phases of the emulsions of embodiments as described herein.

In another embodiment of the invention, the soft gel matrix fill comprises two phases in the form of an emulsion (emulsion-type matrix). The emulsion can be a water-in-oil type emulsion. The internal phase comprises aqueous or hydrophilic materials, such as polyethylene glycol of molecular weight ranging from about 200 to about 8000. The internal phase can also be an aqueous or hydro-alcoholic solution comprising cellulose derivatives, polyacrylates, or polyvinyl polymers. Examples of such polymers include methylcellulose, hydroxypropylmethyl cellulose, polymethylmethacrylate, and polyvinylpyrrolidone (PVP). The internal phase state can be “fluid” or “structured.” A “fluid” internal phase, as used herein, means a completely flowable liquid whose globules can aggregate to make a larger globule. A “structured” internal phase, as used herein, means a solid, semisolid or a gel whose shape is relatively stable and does not usually aggregate to form a large globule. A structured internal phase therefore provides more controlled drug release and stabilizes the physical state of the matrix.

The external phase of the matrix fill emulsion comprises lipid or lipophilic materials similar to those described above. The active ingredient or drug can be dispersed in the internal phase as a solution and/or as a suspension. The emulsion matrix can contain a surfactant or combination of surfactants having HLB values ranging from about 2 to about 20. The HLB range can also be from about 5 to about 15.

In another embodiment, the matrix fill is of an emulsion type, where the drug is distributed in both external and internal phases. One portion of the active ingredient or drug in form of solid particles can be incorporated in the internal phase, while another portion is dispersed in the external phase as solid particles.

This invention also provides methods for making controlled release products in a soft capsule form. The methods are applicable for production of controlled release preparations of low dose (potent) drugs that are highly water-soluble. The methods are also suitable for preparing controlled release products of relatively less potent, moderately water-soluble drugs.

The suspension-type matrix fill compositions can be used for drugs that are moderately water-soluble at a dosage of between about 25 mg to about 500 mg. Such drugs include non-steroid anti-inflammatory drugs and anti-asthmatics, e.g., diclofenac, naproxene, ibuprofen, ketoprofen, celecoxib, and theophylline.

On the other hand, the emulsion-type matrix fill can be used for highly water-soluble molecules such as anti-asthmatics, narcotic analgesics, and analgesic antagonists as well as cardiovascular drugs, e.g., diltiazem, nifidipine, oxycodone, morphine, morphine analogues, and morphine antagonists.

The suspension-type matrix fill can be manufactured by mixing the active ingredient or drug with the lipid or lipophilic matrix ingredients using mechanical or ultrasonic forces. Applying heat while or prior to mixing has the benefit of reducing the matrix viscosity. Reduced matrix viscosity in turn results in more efficient mixing. The matrix materials can be heated to temperature at or close to the melting point of the matrix composite. The melting point of the composite matrix is workable in the range of from about 25° C. to about 70° C. The melting point range of the matrix composition can also be from about 30° C. to about 50° C. The drug-to-matrix ratio can be concentrated enough to provide a low total mass per unit dose, yet can still be flowable to allow encapsulation using a rotary die encapsulation machine. A workable drug-to-matrix ratio range is from about 1:9 to about 1:1 by weight. The drug-to-matrix ratio range can also be from about 1:8 to about 1:1 by weight.

The emulsion-type of matrix fill can be manufactured by dispersing the active ingredient or drug in the internal phase to provide a clear solution or suspension. The active ingredient or drug can be dispersed using propeller or homogenizer mixers. The internal phase materials can then be added to the molten external phase containing surfactant from about 0.1% to about 5% by weight. The emulsion can be made using mechanical forces generated by a propeller mixer, a homogenizer, or a microfluidizer. The matrix is then cooled to a temperature of from about 20° C. to about 35° C. for encapsulation using a rotary die encapsulation machine. The internal-to-external phase workable ratio is in the range of from about 0.5:10 to about 1:1 by weight. The ratio range can also be from about 1:9 to about 1:1 by weight. The workable drug-to-matrix ratio can be from about 1:100 to about 1:2 by weight. The range of the drug-to-matrix can also be from about 1:50 to about 1:3 by weight.

In an alternative aspect, the matrix fills of the invention are encapsulated in hard shell capsules. Guidance regarding hard shell, liquid filling technology can be found in Walker, S. E., et al., “The filling of molten and thixotropic formulations into hard gelatin capsules,” J. Pharm. Pharmacol. 32:389-393 (1980); McTaggert, C., et al., “The evaluation of an automatic system for filling liquids into hard gelatin capsules,” J. Pharm. Pharmacol. 36:119-121 (1984); Hawley, A. R. et al., “Physical and chemical characterization of thermosoftened bases for molten filled hard gelatin capsule formulations,” Drug. Devel. Ind. Pharm. 18(16): 1719 (1992); and Cade, D., et al., “Liquid filled and sealed hard gelatin capsules,” Acta Pharm. Technol. 33(2):97-100 (1987), all fully incorporated herein by reference.

The following Examples are intended for purposes of illustration only, and should not be interpreted as limiting in any way of the scope of the invention.

### EXAMPLES

Formulation 1:

| Ingredients | Amount (% w/w) |
|---|---|
| Diltiazem Hydrochloride | 5.00 |
| Soybean Oil | 6.24 |
| Vegetable Shortening | 60.00 |
| Vegetable Flakes | 12.00 |
| Glyceryl mono oleate | 2.35 |
| Span 60* | 0.16 |
| Methyl Cellulose | 1.50 |
| PEG 3350 | 4.50 |
| PEG 400 | 8.25 |

*sorbitan stearate.

Procedure:

Vegetable shortening, vegetable flakes, Glyceryl mono oleate, Span 60 and soybean oil were melted together at 50° to 70° C. (wax or lipophilic phase). Methylcellulose, PEG 3350 and PEG 400 were melted separately at 50° to 70° C. (aqueous phase). Diltiazem hydrochloride was dispersed in the melted aqueous phase and added slowly to the wax phase with homogenization, while maintaining the temperature between 50° and 70° C. The resultant homogeneous emulsion phase was cooled and encapsulated.

Evaluation:

Filled capsules were subjected to dissolution as per USP using the paddle method in distilled water at 100 RPM.

Result:

T₅₀ (time required for 50% dissolution) is about 18 h.

Note: The Procedure and Evaluation followed for Formulation 1 was also used for Formulations 2-24 below.

Formulation 2:

| Ingredients | Amount (% w/w) |
|---|---|
| Diltiazem Hydrochloride | 5.00 |
| Soybean Oil | 27.84 |
| Vegetable Shortening | 38.40 |
| Vegetable Flakes | 12.00 |
| Glyceryl mono oleate | 2.35 |
| Span 60 | 0.16 |
| Methyl Cellulose | 1.50 |
| PEG 3350 | 4.50 |
| PEG 400 | 8.25 |

Result: T₅₀ (time required for 50% dissolution) is about 3 h.

Formulation 3:

| Ingredients | Amount (% w/w) |
|---|---|
| Diltiazem Hydrochloride | 5.00 |
| Soybean Oil | 23.84 |
| Vegetable Shortening | 42.40 |
| Vegetable Flakes | 12.00 |
| Glyceryl mono oleate | 2.35 |
| Span 60 | 0.16 |
| Methyl Cellulose | 1.50 |
| PEG 3350 | 4.50 |
| PEG 400 | 8.25 |

Result: T₅₀ (time required for 50% dissolution) is about 1 h.

Formulation 4:

| Ingredients | Amount (% w/w) |
|---|---|
| Diltiazem Hydrochloride | 10.00 |
| Soybean Oil | 4.68 |
| Vegetable Shortening | 44.70 |
| Vegetable Flakes | 9.00 |
| Glyceryl mono oleate | 2.70 |
| Span 60 | 0.12 |
| Lecithin | 0.30 |
| Methyl Cellulose | 3.00 |
| PEG 3350 | 9.00 |
| PEG 400 | 16.50 |

Result: T₅₀ (time required for 50% dissolution) is about 4 h.

Formulation 5:

| Ingredients | Amount (% w/w) |
|---|---|
| Diltiazem Hydrochloride | 10.00 |
| Soybean Oil | 20.88 |
| Vegetable Shortening | 25.50 |
| Vegetable Flakes | 12.00 |
| Glyceryl mono oleate | 2.70 |
| Span 60 | 0.12 |
| Lecithin | 0.30 |
| Methyl Cellulose | 3.00 |
| PEG 3350 | 9.00 |
| PEG 400 | 16.50 |

Result: T₅₀ (time required for 50% dissolution) is about 8 h.

Formulation 6:

| Ingredients | Amount (% w/w) |
|---|---|
| Diltiazem Hydrochloride | 10.00 |
| Soybean Oil | 20.88 |
| Vegetable Shortening | 28.50 |
| Vegetable Flakes | 9.00 |
| Glyceryl mono oleate | 2.70 |
| Span 60 | 0.12 |
| Lecithin | 0.30 |
| Methyl Cellulose | 3.00 |
| PEG 3350 | 12.00 |
| PEG 400 | 13.50 |

Result: T₅₀ (time required for 50% dissolution) is about 3.5 h.

Formulation 7:

| Ingredients | Amount (% w/w) |
|---|---|
| Diltiazem Hydrochloride | 10.00 |
| Soybean Oil | 27.00 |
| Vegetable Shortening | 13.88 |
| Vegetable Flakes | 18.00 |
| Glyceryl mono oleate | 2.70 |
| Span 60 | 0.12 |
| Lecithin | 0.30 |
| Methyl Cellulose | 3.00 |
| PEG 3350 | 9.00 |
| PEG 400 | 16.50 |

Result: T₅₀ (time required for 50% dissolution) is about 4 h.

Formulation 8:

| Ingredients | Amount (% w/w) |
|---|---|
| Diltiazem Hydrochloride | 10.00 |
| Soybean Oil | 27.00 |
| Vegetable Shortening | 13.88 |
| Vegetable Flakes | 18.00 |
| Glyceryl mono oleate | 2.70 |
| Span 60 | 0.12 |
| Lecithin | 0.30 |
| Methyl Cellulose | 3.00 |
| PEG 3350 | 12.00 |
| PEG 400 | 13.50 |

Result: T₅₀ (time required for 50% dissolution) is about 11 h.

Formulation 9:

| Ingredients | Amount (% w/w) |
|---|---|
| Diltiazem Hydrochloride | 10.00 |
| Soybean Oil | 23.38 |
| Vegetable Shortening | 24.00 |
| Yellow Beeswax | 6.00 |
| Vegetable Flakes | 6.00 |
| Glyceryl mono oleate | 2.70 |
| Span 60 | 0.12 |
| Lecithin | 0.30 |
| Methyl Cellulose | 3.00 |
| PEG 3350 | 9.00 |
| PEG 400 | 16.50 |

Result: T₅₀ (time required for 50% dissolution) is about 10 h.

Formulation 10:

| Ingredients | Amount (% w/w) |
|---|---|
| Diltiazem Hydrochloride | 10.00 |
| Soybean Oil | 18.65 |
| Vegetable Shortening | 20.00 |
| Yellow Beeswax | 5.00 |
| Vegetable Flakes | 5.00 |
| Glyceryl mono oleate | 3.00 |
| Span 60 | 0.12 |
| Lecithin | 0.30 |
| Methyl Cellulose | 3.00 |
| PEG 3350 | 9.00 |
| PEG 400 | 16.50 |

Result: T₅₀ (time required for 50% dissolution) is about 3.5 h.

Formulation 11:

| Ingredients | Amount (% w/w) |
|---|---|
| Diltiazem Hydrochloride | 10.00 |
| Soybean Oil | 23.38 |
| Vegetable Shortening | 24.00 |
| Yellow Beeswax | 6.00 |
| Vegetable Flakes | 6.00 |
| Glyceryl mono oleate | 2.70 |
| Span 60 | 0.12 |
| Lecithin | 0.30 |
| Methyl Cellulose | 3.00 |
| PEG 3350 | 15.00 |
| PEG 400 | 10.50 |

Result: T₅₀ (time required for 50% dissolution) is about >24 h.

Formulation 12:

| Ingredients | Amount (% w/w) |
|---|---|
| Diltiazem Hydrochloride | 10.00 |
| Soybean Oil | 18.65 |
| Vegetable Shortening | 20.00 |
| Yellow Beeswax | 5.00 |
| Vegetable Flakes | 5.00 |
| Glyceryl mono oleate | 3.00 |
| Span 60 | 0.12 |
| Lecithin | 0.30 |
| Methyl Cellulose | 3.00 |
| PEG 3350 | 15.00 |
| PEG 400 | 10.50 |

Result: T₅₀ (time required for 50% dissolution) is about >24 h.

Formulation 13:

| Ingredients | Amount (% w/w) |
|---|---|
| Diltiazem Hydrochloride | 10.00 |
| Soybean Oil | 10.39 |
| Vegetable Shortening | 31.99 |
| Yellow Beeswax | 8.00 |
| Vegetable Flakes | 8.00 |
| Glyceryl mono oleate | 2.70 |
| Span 60 | 0.12 |
| Lecithin | 0.30 |
| Methyl Cellulose | 3.00 |
| PEG 3350 | 9.00 |
| PEG 400 | 16.50 |

Result: T₅₀ (time required for 50% dissolution) is about 6.5 h.

Formulation 14:

| Ingredients | Amount (% w/w) |
|---|---|
| Diltiazem Hydrochloride | 10.00 |
| Soybean Oil | 8.66 |
| Vegetable Shortening | 26.67 |
| Yellow Beeswax | 6.67 |
| Vegetable Flakes | 6.67 |
| Glyceryl mono oleate | 3.00 |
| Span 60 | 0.10 |
| Lecithin | 0.25 |
| Methyl Cellulose | 4.00 |
| PEG 3350 | 12.00 |
| PEG 400 | 22.00 |

Result: T₅₀ (time required for 50% dissolution) is about 3.5 h.

Formulation 15:

| Ingredients | Amount (% w/w) |
|---|---|
| Diltiazem Hydrochloride | 10.00 |
| Soybean Oil | 10.34 |
| Vegetable Shortening | 32.00 |
| Yellow Beeswax | 8.00 |
| Vegetable Flakes | 8.00 |
| Glyceryl mono oleate | 2.50 |
| Span 60 | 0.10 |
| Lecithin | 0.30 |
| Methyl Cellulose | 3.00 |
| PEG 3350 | 15.00 |
| PEG 400 | 10.50 |

Result: T₅₀ (time required for 50% dissolution) is about >24 h.

Formulation 16:

| Ingredients | Amount (% w/w) |
|---|---|
| Diltiazem Hydrochloride | 10.00 |
| Soybean Oil | 8.66 |
| Vegetable Shortening | 26.67 |
| Yellow Beeswax | 6.67 |
| Vegetable Flakes | 6.67 |
| Glyceryl mono oleate | 3.00 |
| Span 60 | 0.10 |
| Lecithin | 0.25 |
| Methyl Cellulose | 4.00 |
| PEG 3350 | 20.00 |
| PEG 400 | 14.00 |

Result: T₅₀ (time required for 50% dissolution) is about 6.5 h.

Formulation 17:

| Ingredients | Amount (% w/w) |
|---|---|
| Diltiazem Hydrochloride | 10.00 |
| Soybean Oil | 46.34 |
| Vegetable Shortening | 8.00 |
| Yellow Beeswax | 2.00 |
| Vegetable Flakes | 2.00 |
| Glyceryl mono oleate | 2.70 |
| Span 60 | 0.12 |
| Lecithin | 0.30 |
| Methyl Cellulose | 3.00 |
| PEG 3350 | 15.00 |
| PEG 400 | 10.50 |

Result: T₅₀ (time required for 50% dissolution) is about 1.5 h.

Formulation 18:

| Ingredients | Amount (% w/w) |
|---|---|
| Diltiazem Hydrochloride | 10.00 |
| Soybean Oil | 38.66 |
| Vegetable Shortening | 6.67 |
| Yellow Beeswax | 1.67 |
| Vegetable Flakes | 1.67 |
| Glyceryl mono oleate | 3.00 |
| Span 60 | 0.10 |
| Lecithin | 0.25 |
| Methyl Cellulose | 4.00 |
| PEG 3350 | 20.00 |
| PEG 400 | 14.00 |

Result: T₅₀ (time required for 50% dissolution) is about 1.5 h.

Formulation 19:

| Ingredients | Amount (% w/w) |
|---|---|
| Diltiazem Hydrochloride | 10.00 |
| Soybean Oil | 34.34 |
| Vegetable Shortening | 16.00 |
| Yellow Beeswax | 4.00 |
| Vegetable Flakes | 4.00 |
| Glyceryl mono oleate | 2.70 |
| Span 60 | 0.12 |
| Lecithin | 0.30 |
| Methyl Cellulose | 3.00 |
| PEG 3350 | 15.00 |
| PEG 400 | 10.50 |

Result: T₅₀ (time required for 50% dissolution) is about 20 h.

Formulation 20:

| Ingredients | Amount (% w/w) |
|---|---|
| Diltiazem Hydrochloride | 10.00 |
| Soybean Oil | 28.66 |
| Vegetable Shortening | 13.33 |
| Yellow Beeswax | 3.33 |
| Vegetable Flakes | 3.33 |
| Glyceryl mono oleate | 3.00 |
| Span 60 | 0.10 |
| Lecithin | 0.25 |
| Methyl Cellulose | 4.00 |
| PEG 3350 | 20.00 |
| PEG 400 | 14.00 |

Result: T₅₀ (time required for 50% dissolution) is about 20 h.

Formulation 21:

| Ingredients | Amount (% w/w) |
|---|---|
| Diltiazem Hydrochloride | 5.00 |
| Soybean Oil | 12.46 |
| Vegetable Shortening | 52.50 |
| Vegetable Flakes | 3.50 |
| Glyceryl mono oleate | 2.65 |
| Span 60 | 0.20 |
| Methyl Cellulose | 2.50 |
| PEG 900 | 15.75 |
| PEG 400 | 5.25 |

Result: T₅₀ (time required for 50% dissolution) is about 0.3 h.

Formulation 22:

| Ingredients | Amount (% w/w) |
|---|---|
| Diltiazem Hydrochloride | 5.00 |
| Soybean Oil | 9.79 |
| Vegetable Shortening | 27.50 |
| Vegetable Flakes | 2.75 |
| Glyceryl mono oleate | 2.75 |
| Glyceryl mono stearate | 2.00 |
| Span 60 | 1.00 |
| Methyl Cellulose | 4.00 |
| PEG 900 | 8.40 |
| PEG 400 | 25.20 |

Result: T₅₀ (time required for 50% dissolution) is about 0.3 h.

Formulation 23:

| Ingredients | Amount (% w/w) |
|---|---|
| Famotidine | 1.00 |
| Soybean Oil | 12.00 |
| Vegetable Shortening | 15.00 |
| Vegetable Flakes | 1.50 |
| Glyceryl mono oleate | 1.50 |
| Span 60 | 0.06 |
| Methyl Cellulose | 6.90 |
| Cremophor RH 40 | 0.69 |
| Glyceryl mono stearate | 3.45 |
| PEG 400 | 57.96 |

Result: T₅₀ (time required for 50% dissolution) is about 0.6 h.

Formulation 24:

| Ingredients | Amount (% w/w) |
|---|---|
| Vegetable Shortening | 25.00 |
| Methyl Cellulose | 11.30 |
| Cremophor RH 40 | 0.70 |
| Glyceryl mono stearate | 3.50 |
| PEG 400 | 59.50 |

Formulation 25 (Dual Release):

| Ingredients | Amount (% w/w) |
|---|---|
| Diltiazem Hydrochloride | 10.33 |
| Soybean Oil | 36.15 |
| Vegetable Shortening | 10.74 |
| Yellow Beeswax | 2.69 |
| Vegetable Flakes | 2.69 |
| Glyceryl mono oleate | 2.87 |
| Span 60 | 0.11 |
| Lecithin | 0.27 |
| Methyl Cellulose | 3.60 |
| PEG 3350 | 17.98 |
| PEG 400 | 12.59 |

Procedure:

Vegetable shortening, vegetable flakes, yellow beeswax, glyceryl mono oleate, lecithin, Span 60 and soybean oil were melted together at 50° to 70° C. (wax phase). Methylcellulose, PEG 3350 and PEG 400 were melted separately at 50° to 70° C. (aqueous phase). About 77% of diltiazem hydrochloride was dispersed in the melted aqueous phase and added slowly to the wax phase with homogenization, while maintaining the temperature between 50° and 70° C. Remaining 23% of diltiazem hydrochloride was added to the final resultant homogeneous emulsion. The emulsion was cooled and encapsulated.

Evaluation:

Filled capsules were subjected for dissolution as per USP using paddle method in distilled water at 100 RPM.

Result: T₅₀ (time required for 50% dissolution) is about 4.2 h.

Formulation 26:

| Ingredients | Amount (% w/w) |
|---|---|
| Oxycodone Hydrochloride | 5.00 |
| Soybean Oil | 36.56 |
| Vegetable Shortening | 11.00 |
| Yellow Beeswax | 2.75 |
| Vegetable Flakes | 2.75 |
| Glyceryl mono oleate | 3.35 |
| Span 60 | 0.55 |
| Lecithin | 0.28 |
| Methyl Cellulose | 4.00 |
| PEG 3350 | 20.00 |
| PEG 400 | 14.00 |

Procedure & Evaluation: Procedure adopted was as described in Formulation 1.

Result: T₅₀ (time required for 50% dissolution) is about 3.5 h.

Formulation 27:

| Ingredients | Amount (% w/w) |
|---|---|
| Oxycodone Hydrochloride | 5.00 |
| Water | 6.00 |
| Soybean Oil | 36.56 |
| Vegetable Shortening | 11.00 |
| Yellow Beeswax | 2.75 |
| Vegetable Flakes | 2.75 |
| Glyceryl mono oleate | 3.10 |
| Span 60 | 0.55 |
| Lecithin | 0.28 |
| Methyl Cellulose | 4.00 |
| PEG 3350 | 20.00 |
| PEG 400 | 8.00 |

Procedure:

Procedure adopted was similar to Formulation 25, but the model drug was dissolved in water before adding to the rest of the formulation.

Evaluation:

Filled capsules were subjected for dissolution as per USP using paddle method in distilled water at 100 RPM.

Result: T₅₀ (time required for 50% dissolution) is about >8 h.

Formulation 28:

| Ingredients | Amount (% w/w) |
|---|---|
| Theophylline | 10.00 |
| Soybean Oil | 36.36 |
| Vegetable Shortening | 45.45 |
| Vegetable Flakes | 3.64 |
| Glyceryl Mono oleate | 4.54 |
| Cremophor EL 40 | 0.91 |

Procedure:

Vegetable shortening, vegetable flakes, GMO, and Cremophor EL 40 were melted with soybean oil between 50 and 70° C. To this melted mass, theophylline was added and homogenized. The resultant mixture was cooled while mixing and encapsulated.

Result: T₅₀ (time required for 50% dissolution) is about 1 h.

Formulation 29:

| Ingredients | Amount (% w/w) |
|---|---|
| Theophylline | 10.00 |
| Soybean Oil | 36.36 |
| Vegetable Shortening | 45.45 |
| Vegetable Flakes | 4.32 |
| Glyceryl Mono oleate | 4.54 |
| Cremophor RH 40 | 0.23 |

Procedure: Procedure adopted was similar to Formulation 28.

Result: T₅₀ (time required for 50% dissolution) is about >24 h.

Formulation 30:

| Ingredients | Amount (% w/w) |
|---|---|
| Theophylline | 10.00 |
| Soybean Oil | 36.36 |
| Vegetable Shortening | 45.45 |
| Vegetable Flakes | 3.86 |
| Glyceryl Mono oleate | 4.54 |
| Cremophor RH 40 | 0.68 |

Procedure: Procedure adopted was similar to Formulation 28.

Result: T₅₀ (time required for 50% dissolution) is about 16 h.

Formulation 31:

| Ingredients | Amount (% w/w) |
|---|---|
| Theophylline | 10.00 |
| Soybean Oil | 36.36 |
| Vegetable Shortening | 45.45 |
| Vegetable Flakes | 4.09 |
| Glyceryl Mono oleate | 4.54 |
| Cremophor RH 40 | 0.45 |

Procedure: Procedure adopted was similar to Formulation 28.
Result: T₅₀ (time required for 50% dissolution) is about 12 h.

1-11. (canceled)

12. A controlled release soft capsule having a shell and a matrix fill comprising an active ingredient or drug, wherein the matrix fill comprises two phases in the form of an emulsion.

13. The controlled release soft capsule of claim 12, wherein the emulsion is a water-in-oil type emulsion.

14. The controlled release soft capsule of claim 12, wherein the emulsion comprises a surfactant or combination of surfactants having HLB values ranging from about 2 to about 20.

15. The controlled release soft capsule of claim 12, wherein the emulsion comprises a surfactant or combination of surfactants having HLB values ranging from about 5 to about 15.

16. The controlled release soft capsule of claim 12, wherein the active ingredient or drug is selected from the group consisting of anti-asthmatics, narcotic analgesics, narcotic antagonists, and cardiovascular drugs.

17. The controlled release soft capsule of claim 12, wherein the active ingredient or drug is selected from the group consisting of diltiazem, nifidipine, oxycodone, morphine, morphine analogues, and morphine antagonists.

18. The controlled release soft capsule of claim 12, wherein the ratio of the active ingredient or drug to the matrix fill is from about 1:100 to about 1:2 by weight.

19. The controlled release soft capsule of claim 12, wherein the ratio of the active ingredient or drug to the matrix fill is from about 1:50 to about 1:3 by weight.

20. The controlled release soft capsule of claim 12, wherein the emulsion comprises an aqueous or hydrophilic internal phase and a lipid or lipophilic external phase.

21. The controlled release soft capsule of claim 20, wherein the internal phase comprises polyethylene glycol of molecular weight ranging from about 200 to about 8000.

22. The controlled release soft capsule of claim 20, wherein the internal phase is an aqueous or hydro-alcoholic solution comprising cellulose derivatives, polyacrylates, polyvinyl polymers, or combinations thereof.

23. The controlled release soft capsule of claim 20, wherein the internal phase comprises at least one polymer selected from the group consisting of methylcellulose, hydroxypropylmethyl cellulose, polymethylmethacrylate, and polyvinylpyrrolidone (PVP).

24. The controlled release soft capsule of claim 20, wherein the internal phase is structured.

25. The controlled release soft capsule of claim 20, wherein the external phase comprises a vegetable oil, hydrogenated vegetable oil, fatty acid, wax, fatty acid ester, or a combination thereof.

26. The controlled release soft capsule of claim 20, wherein the active ingredient or drug is dispersed in the internal phase as a solution or suspension form.

27. The controlled release soft capsule of claim 20, wherein the ratio of the internal phase to external phase is from about 0.5:10 to about 1:1 by weight.

28. The controlled release soft capsule of claim 20, wherein the ratio of the internal phase to external phase is from about 1:9 to about 1:1 by weight.

29. A The controlled release soft capsule of claim 12, wherein the active ingredient or drug is distributed in both an external and internal phase.

30. The controlled release soft capsule of claim 29, wherein the active ingredient or drug is in the form of solid particles.

31. The controlled release soft capsule of claim 29, wherein the active ingredient or drug is present as solid particles incorporated in both the internal phase and the external phase.

32. A controlled release hard shell capsule comprising a shell and a matrix fill, wherein the matrix fill comprises an active ingredient or drug incorporated as solid particles in lipid or lipophilic materials.

33. The controlled release hard shell capsule of claim 32, wherein the lipid or lipophilic material is selected from the group consisting of a vegetable oil, hydrogenated vegetable oil, fatty acid, wax, fatty acid ester, and combinations thereof.

34. The controlled release hard shell capsule of claim 32, wherein the matrix fill comprises a release regulator selected from the group consisting of a fatty acid salt, fatty acid ester, and a fatty acid polyoxyethylene derivative.

35. The controlled release hard shell capsule of claim 34, wherein the release regulator is a surfactant having an hydrophilic/lipophilic balance (HLB) value between about 3 and about 40.

36. The controlled release hard shell capsule of claim 32, wherein the active ingredient or drug is a non-steroid anti-inflammatory drug or an anti-asthmatic.

37. The controlled release hard shell capsule of claim 32, wherein the active ingredient or drug is selected from the group consisting of diclofenac, naproxene, ibuprofen, ketoprofen, celecoxib, or theophylline.

38. The controlled release hard shell capsule of claim 32, wherein the ratio of the active ingredient or drug to the matrix fill is from about 1:9 to about 1:1 by weight.

39. The controlled release hard shell capsule of claim 32, wherein the ratio of the active ingredient or drug to the matrix fill is from about 1:8 to about 1:1 by weight.

40. The controlled release hard shell capsule of claim 32, wherein the physical state of the matrix is a semi-fluid or a structured solid state.

41. The controlled release hard shell capsule of claim 40, wherein the matrix is a fluid or semi-fluid at room temperature or at a body temperature of a subject to which the capsule is intended to be administered.

42. The controlled release hard shell capsule of claim 40, wherein the active ingredient or drug is partially soluble in the matrix and at least a portion of the active ingredient or drug is in solid form in the matrix.

43. A method of manufacturing a matrix fill for a controlled release soft capsule, the method comprising
a) applying heat to matrix components during mixing or prior to mixing at about the melting point of the matrix fill composition; and
b) mixing the active ingredient or drug with the lipid or lipophilic matrix ingredients using mechanical or ultrasonic forces to form the matrix fill.

44. The method of claim 43, wherein the matrix fill is flowable such that it can be encapsulated using a rotary die encapsulation machine.

45. The method of claim 43, wherein the matrix components are heated to a temperature in the range of from about 25° C. to about 70° C.

46. The method of claim 43, wherein the matrix components are heated to a temperature in the range of from about 30° C. to about 50° C.

47. A method of manufacturing a controlled release soft capsule, wherein the matrix fill includes two phases in the form of an emulsion, the method comprising
a) dispersing the active ingredient or drug in an internal phase to form a clear solution or suspension using propeller or homogenizer mixers;
b) adding the internal phase to a molten external phase containing at least one surfactant in an amount from about 0.1% to about 5% by weight to form a resulting mixture;
c) forming an emulsion from the resulting mixture by subjecting the mixture to mechanical forces generated by a propeller mixer, a homogenizer, or a microfluidizer;
d) cooling the emulsion to from about 20° C. to about 35° C.; and
e) encapsulating the emulsion using a rotary die encapsulation machine to form the controlled release capsule.
